(19) United States
(12) Patent Application Publication
(10) Pub. No.: US 2025/0130162 A1
(43) Pub. Date: Apr. 24, 2025

(54) CHARGE-TO-DIGITAL CONVERTER FOR TIME-DOMAIN DUAL LIFETIME REFERENCING

(71) Applicant: Worcester Polytechnic Institute, Worcester, MA (US)

(72) Inventors: Ulkuhan Guler, Shrewsbury, MA (US); Tuna B. Tufan, Worcester, MA (US); John A. McNeill, Stow, MA (US)

(21) Appl. No.: 18/924,362

(22) Filed: Oct. 23, 2024

Related U.S. Application Data
(60) Provisional application No. 63/545,273, filed on Oct. 23, 2023.

(51) Int. Cl. G01N 21/31; G01N 33/00

(52) U.S. Cl. CPC G01N 21/3151; G01N 33/004; G01N 33/0068; G01N 2021/3144

(57) ABSTRACT

A wearable, miniaturized wireless device provides accurate measurement of transcutaneous carbon dioxide diffusing though the skin detects a luminescent response of a carbon dioxide-sensitive film, providing an accurate reflection of a person's blood carbon dioxide levels. The device employs a charge-to-digital converter (CDC) architecture operable for implementation of a time-domain dual lifetime referencing computation to measure transcutaneous carbon dioxide. This potential product enables highly accurate and precise measurements of transcutaneous carbon dioxide while minimizing interference from confounding factors.

### RELATED APPLICATIONS

This patent application claims the benefit under 35 U.S.C. § 119 (e) of U.S. Provisional Patent App. No. 63/545,273, filed Oct. 23, 2023, entitled “TRANSCUTANEOUS GASEOUS MEASUREMENT DEVICE,” incorporated herein by reference in entirety.

### STATEMENT REGARDING FEDERALLY SPONSORED RESEARCH AND DEVELOPMENT

This invention was developed with U.S. Government Support under contract No. 2143898, awarded by the National Science Foundation (NSF). The Government has certain rights in the Invention.

### BACKGROUND

Human respiratory parameters, particular blood gas levels, are key indicators of the physiological condition of the human body. Quantification of the real-time dynamics and physiological distribution of blood gas measurements of carbon dioxide (CO₂) and oxygen (O₂) are important to clinicians to understand the mechanisms associated with both pathological and normal physiological conditions.

### SUMMARY

A wearable, miniaturized wireless device provides accurate measurement of transcutaneous carbon dioxide diffusing though the skin by detecting a luminescent response of a carbon dioxide-sensitive film, providing an accurate reflection of a person's blood carbon dioxide levels. The device employs a charge-to-digital converter (CDC) architecture operable for implementation of a time-domain dual lifetime referencing computation to measure transcutaneous carbon dioxide. This approach enables highly accurate and precise measurements of transcutaneous carbon dioxide while minimizing interference from confounding factors.

In an example configuration, the gaseous measurement device includes a sensing film having an emissive response based on transcutaneous carbon dioxide (PtcCO₂), and a light source disposed for directing pulsed light at the film defined by a sensory planar material suitable for epidermal contact. An adjacent sensor receives re-emitted light from the sensing film, and a monitoring circuit identifies a carbon dioxide-sensitive component of a photocurrent response to the sensing. A charge-to-digital converter for time-domain dual lifetime referencing film following settling of a luminescence from a pulse of the pulsed light. The light source irradiates a blue light, and the monitoring circuit includes a charge-to-digital converter (CDC) used to derive carbon dioxide levels from luminescence data using a time-domain dual lifetime referencing approach.

Configurations herein are based, in part, on the observation that conventional approaches to blood gas or transcutaneous gas measurements suffer from the shortcoming of a general non-portability of accurate measurement devices. Conventional approaches suffer from the shortcoming of heat-based approaches, imposing substantial power requirements, and requiring a tethered connection to an appurtenant computing and measurement device. Accordingly, configurations herein substantially overcome the size, heating and power requirements of conventional approaches to accurately and precisely measure transcutaneous carbon dioxide or other sensed gas in the presence of a sensing film placed or adhered directly on the skin. A proposed circuit design is employed in a miniaturized wireless device for monitoring transcutaneous carbon dioxide, referring to carbon dioxide that diffuses through the skin. This device detects the luminescent response of a carbon dioxide-sensitive film, providing an accurate reflection of a person's blood carbon dioxide levels.

In further detail, configurations below disclose a circuit and system implemented by a blood gas measurement device using a sensing film having an emissive response to a sensed gas, and a light source, where the sensing film has an emissive response to the light source based on a gaseous presence of the sensed gas. A photodetector receives the emissive response from the sensing film, and a sensing circuit is responsive to the photodetector for computing: i: a first digital signal indicative of the sensed gas; and ii: a second digital signal indicative of a reference intensity, such that a concentration of the sensed gas is based on a ratio of the first digital signal and the second digital signal.

In operation, the method of detecting carbon dioxide includes disposing the sensing film in communication with a gaseous source, such as a patient epidermal surface, where the sensing film has an emissive response to a sensed gas, and directing a light source at the sensing film. The sensing film is selected to be responsive to the light source at a wavelength for generating the emissive response. A photodetector receives the emissive response from a plurality of luminophore types in the sensing film. Upon receiving the emissive response in response to a pulsed light source, a sensing circuit measures a respective digital signal from each of the plurality of luminophore types based on the emissive response, and computes, based on a difference in the plurality of digital signals, a concentration of the sensed gas.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a system context diagram including a wearable sensor suitable for use with configurations herein;

FIG. 2 shows a luminescent response of a sensing film exhibiting a dual lifetime response of respective luminophores in the sensing film of the wearable sensor of FIG. 1;

FIGS. 3A-3C show luminescence curves for excitation and decay of the respective luminophores in the sensing film of FIG. 2;

FIG. 4 shows a circuit schematic of a sensing circuit for analyzing the luminescent response of FIGS. 3A-3C;

FIG. 5 shows a graphical representation of the integral values varied by the gaseous presence of carbon dioxide during operation of the sensing circuit of FIG. 4; and

FIGS. 6A and 6B depict operational trials and verification of the disclosed approach.

### DETAILED DESCRIPTION

The description below presents an example sensing circuit and method to accurately and precisely measure transcutaneous carbon dioxide, which refers to the carbon dioxide that diffuses through the skin. The proposed circuit design will be employed in a miniaturized wireless device for monitoring transcutaneous carbon dioxide. Other gaseous responses may be analyzed based on the sensing film. The example circuit and device detects the luminescent response of a carbon dioxide-sensitive film, providing an accurate reflection of a person's blood carbon dioxide levels.

The example configuration below demonstrates utilization of a miniaturized wireless transcutaneous carbon dioxide monitor incorporating the proposed circuit design for remotely monitoring the respiratory conditions of these patients beyond the confines of a clinical environment. In clinical settings, this advancement enhances the possibility of early patient discharge and reduces the risk of undiagnosed medical issues worsening after leaving the hospital. This represents a substantial improvement over conventional bulky bedside blood gas monitors.

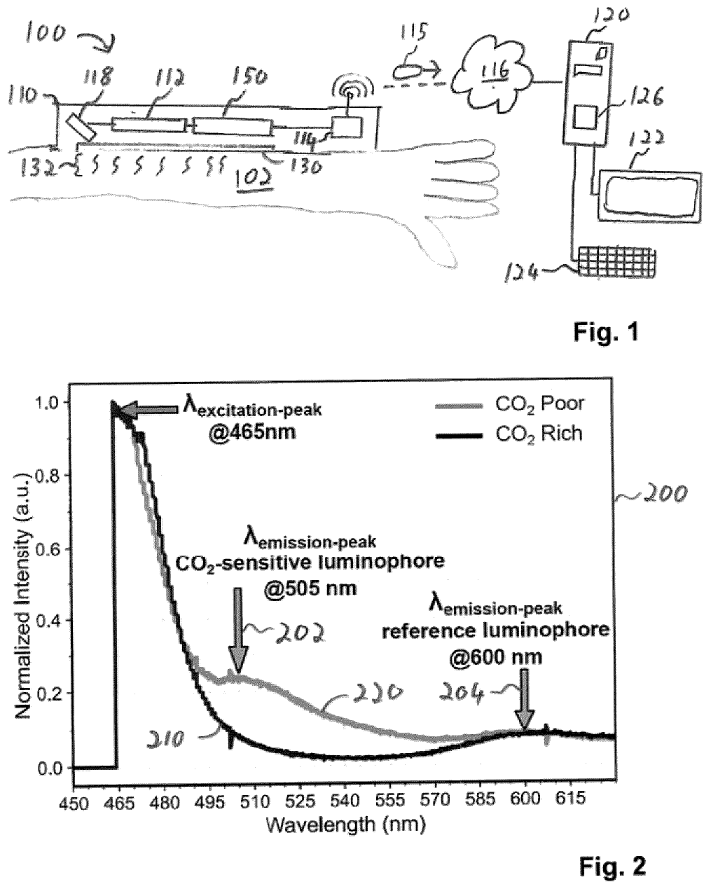

FIG. 1 is a system context diagram including a wearable sensor suitable for use with configurations herein. Referring to FIG. 1, in an example use case in an ambulatory patient environment 100, the gaseous measurement device 110 includes the sensing film 130, sensing circuit 150, a power supply 112, a transmitter 114 and a light source 118, such as an LED.

The gaseous measurement device 110 has a portable and lightweight form factor suitable for adherence or mounting on a patient 102 arm, wrist or other dermal surface. The sensing film 130 is disposed in the presence of the sensed gas 132, whether carbon dioxide or other gaseous presence. The transmitter 114 transmits output data 115 from the sensing circuit 150 to a measurement server 120 via a network 116 using Bluetooth®, WiFi® or other suitable wireless network and transport medium. The measurement server 120 includes analysis logic 126 for processing and rendering the output data 115 as described herein via a visual rendering device 122 and keyboard 124, or other suitable rendering and/or storage medium.

The sensing circuit 150 implements a charge-to-digital converter architecture that is specifically designed for the implementation of the time-domain dual lifetime referencing computation to measure transcutaneous carbon dioxide. Transcutaneous carbon dioxide, defined by the partial pressure of the carbon dioxide that diffuses through the skin, will be sensed by using a commercial carbon dioxide-sensitive film, or sensing film 130. The sensing film 130 emits light in two different peak wavelengths in response to a blue light excitation, as depicted in FIG. 2.

FIG. 2 shows a luminescent response 200 of the sensing film 130 exhibiting a dual lifetime response of respective luminophores in the sensing film 130 of a wearable sensor defined by the gaseous measurement device 110 of FIG. 1. The sensing film 130 is typically a flexible film encased in a plastic or polymer material for humidity protection and longevity of the sensory capabilities. Referring to FIGS. 1 and 2, a first emission peak 202 in the sensing film 130 occurs at 505 nm, produced by the luminescence of the carbon dioxide-sensitive luminophores, while the reference luminophores reach the peak luminescence at 600 nm (204). When carbon dioxide is introduced into the sensing film, the intensity of the luminescence from carbon dioxide-sensitive luminophores decreases, as shown by the CO₂ rich response 210, in contrast to the CO₂ poor response 220. However, the intensity of luminescence from the reference luminophores remains generally unaffected by the change in carbon dioxide. Another difference between the carbon dioxide-sensitive and reference luminophores lies in their respective lifetimes. The carbon dioxide-sensitive luminophores typically have lifetimes measured in nanoseconds, indicating the average duration it takes for them to revert to their ground state after being excited. In contrast, the reference luminophores have lifetimes measured in microseconds, signifying a much longer average duration for their return to the ground state upon excitation. This duality in lifetime enables an approach to sense carbon dioxide changes amendable to the time-domain dual lifetime referencing approach.

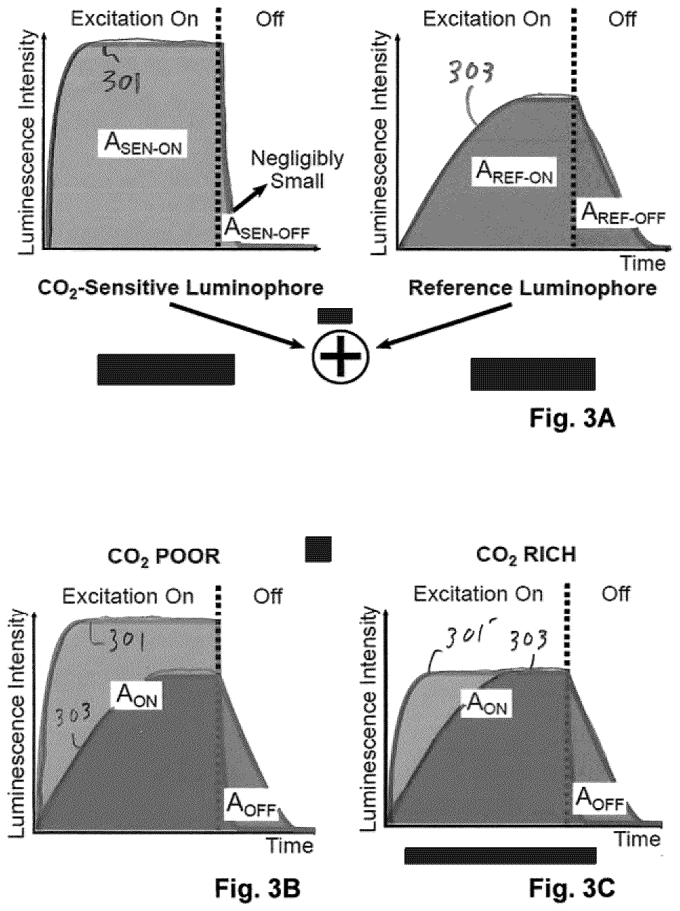

FIGS. 3A-3C show luminescence curves for excitation and decay of the respective luminophores in the sensing film 130 of FIG. 2. A core concept behind the time-domain dual lifetime referencing is to produce a measurement outcome that is immune to external factors affecting both luminescent responses. FIGS. 3A-3C illustrate the luminescence intensity of each component as a function of time when the sensing film is excited with a pulse of blue light under carbon dioxide-lean or poor, and carbon dioxide-rich conditions. FIG. 3A depicts an aggregate luminescence A_{SEN-ON} (301) resulting from the sensory luminophores during excitation. FIG. 3B depicts an aggregate luminescence A_{REF-ON} (303) resulting from the reference luminophores during excitation. The total luminescence during the excitation, A_{ON} depends on the luminescence of both luminophores (A_{SEN-ON} and A_{REF-ON}), confounding factors, and the carbon dioxide level, as shown in FIGS. 3B and 3C. Conversely, the total luminescence following the excitation, referred to as A_{OFF}, is solely determined by the luminescence of the reference luminophore (A_{REF-OFF}). This occurs because when the excitation source is deactivated, the luminescent emissions of the luminophores diminish at a rate proportional to their respective lifetimes. Given that the carbon dioxide-sensitive luminophore has a very short lifetime in the nanosecond range, its luminescence decreases rapidly, making A_{SEN-OFF} negligible when compared to A_{REF-OFF}. Additionally, other confounding factors also contribute to A_{OFF}. By taking the ratio, A_{ON}/A_{OFF}, which is designated as the luminescence ratio, the sensing circuit 150 obtains a measurement of carbon dioxide that is independent of the confounding factors, including excitation source strength and detector photosensitivity.

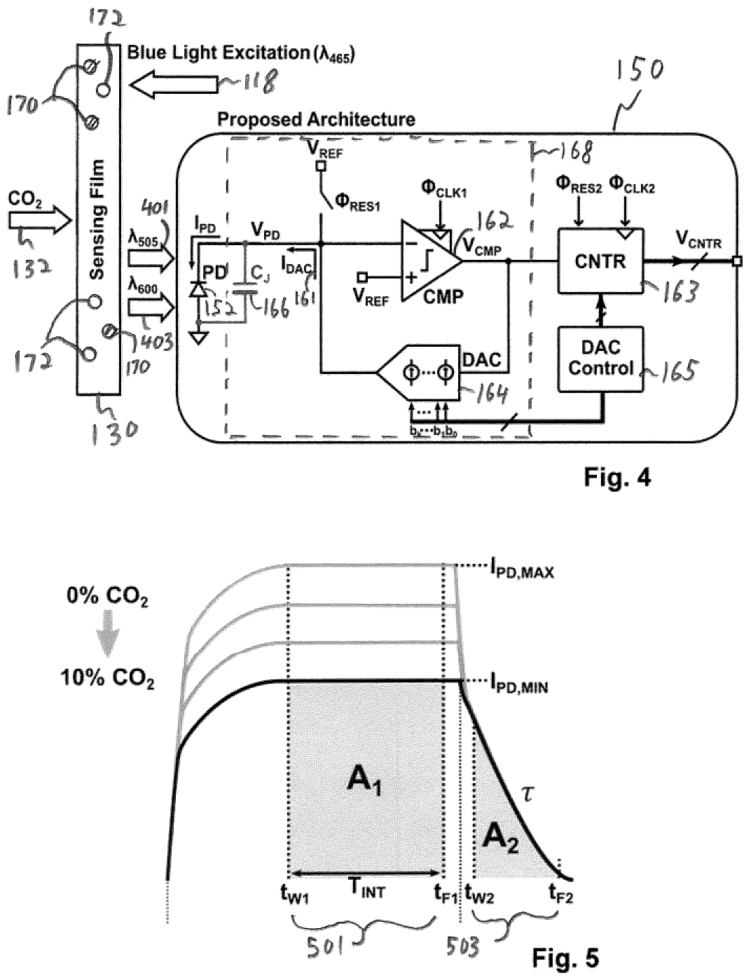

FIG. 4 shows a circuit schematic of a sensing circuit 150 for analyzing the luminescent response of FIGS. 3A-3C. Recalling from FIG. 1, the gaseous measurement device 110 includes a sensing film 130 having an emissive response to the sensed gas 132 and the light source 118, where the sensing film 130 has a dual response to the light source 118 based on a gaseous presence of the sensed gas 132. A photodetector 152 receives the emissive response from the sensing film 130. The sensing circuit 150 is responsive to the photodetector 152 for computing:
i: a first digital signal 301 indicative of the sensed gas 132 (FIG. 3A) during the excitation phase;
ii: a second digital signal 303 indicative of a reference intensity (FIG. 3B), such that a concentration of the sensed gas 132 is based on a ratio of the first digital signal and the second digital signal. The response corresponding to the sensed gas varies with a CO₂ poor environment 301 and a CO₂ rich environment 301′ in FIG. 3C. The first and second digital signals are effectively comingled in the total aggregated signal during the excitation phase, and during the decay phase the second digital signal (reference) dominates as the sensory component decays rapidly.

FIG. 4 depicts the sensing circuit 150 including the charge-to-digital converter (CDC) 168 used to derive carbon dioxide levels from luminescence data using the time-domain dual lifetime referencing approach. The luminescence is captured by the photodetector 152, which in the example configuration is a photodiode with a spectral range that covers the wavelengths of luminescence. The photodiode current (I_{PD}) is composed of the luminescence of the carbon dioxide-sensitive (λ₅₀₅) 401 and reference components (λ₆₀₀). I_{PD} 403, in response to a pulsed excitation, shown below in FIG. 5.

In the sensor circuit 150, the first digital signal 301 is indicative of: the emissive response from the sensing film 130 representing both an emissive component from the sensed gas 132 and an emissive component from the reference emission. the second digital signal 303 represents only an emissive component based on the reference emission, and thus invariant with respect to the carbon dioxide presence. As a reference signal, however, it normalizes other factors such as temperature, the light source 110 brightness and other factors such that the aggregate difference in the emissions from the respective luminophore types is indicative of the carbon dioxide. Sensing films with luminophores sensitive to other substances can also be similarly employed.

The sensing film 130 is therefore selected to include sensory luminophores 170 responsive to the light source 118 for emitting a response intensity based on a presence of the sensed gas 132, and reference luminophores 172 responsive to the light source 118 for emitting a response intensity agnostic to the presence of the sensed gas 132. In the sensing film 130, the sensory luminophores 170 are responsive to a light at a predetermined wavelength for emitting the emissive response at a sensory wavelength with an intensity indicative of the gaseous presence of the sensed gas 132. The reference luminophores 172 are responsive to the light for emitting the emissive response at a reference wavelength different than the sensory wavelength, shown in FIG. 1.

The sensing circuit 150 defines the charge to digital converter 168 that performs an effective integration of the respective areas under the curves 301, 303 of FIGS. 3A-3C for determining the presence and concentration or quantity of the sensed gas 132 stimulating the sensory luminophores 170 in contrast to the reference luminophores 172, allowing an accurate measurement of the sensed gas 132 independently of other ambient factors and without the need for heat or bulky, power consuming circuitry.

The sensing circuit 150 includes the charge to digital converter 168, which further comprises the photodetector 152 disposed for receiving sensory emissions 401 and reference emissions 403 from the sensing film 130 resulting from excitation by the light source 118. A comparator 162 connects to the photodetector 152 for receiving a voltage signal indicative of the sensed gas 132. A digital to analog converter 164 (DAC) connects to an output of the comparator 162 and is configured for generating a feedback current 161 responsive to an output from the comparator 162. The photodetector has an inherent capacitance often called parasitic or junction capacitance, shown as a capacitor 166. Typically this is not a separate discrete element, however an external capacitor could be applied. The capacitor 166 is connected in parallel with the photodetector 152, such that the capacitor 166 receives the feedback current 161 for integrating an aggregate signal received from the photodetector 152 based on the emissive response (emissions 401, 403).

A counter 163 also connects to the output of the comparator 162 for generating a count proportional to the emissive response from the sensing film 130 based on both the sensed gas 132, from emission 401, and the reference intensity, emission 403. The sensory luminophores 170 have an emissive response with an intensity varied by a presence of the sensed gas 132, such as carbon dioxide. This property of the sensing film 130 provides that the first digital signal is indicative of a photodetector 152 current responsive to a pulsed illumination based on an emissive intensity from both the sensory luminophores 170 and the reference luminophores, and the second digital signal is indicative of a photodetector 152 current responsive to the pulsed illumination based on an emissive intensity from the reference luminophores 172. It is equally apparent that the photodetector 152 is selected for responsiveness to light at both the sensory wavelength and the reference wavelength.

The underlying operation of the charge to digital converter 168 is to integrate the charge induced by the photocurrent at the junction capacitance of the photodiode (C_{J}) and maintain the voltage (V_{PD}) at the integration node around an external reference voltage (V_{REF}) using the comparator 162 and the digital-to-analog converter 164 (DAC) that defines a current feedback to the integration node based on the level of V_{PD}. In this sense the sensing circuit is a delta-sigma modulator-based CDC. The counter 163 keeps track of how many times V_{PD} surpasses V_{REF} during the modulation phase, essentially counting the high outputs from the comparator 162 (V_{CMP}). This count is directly proportional to the number of active DAC bits involved in the comparison process, based on DAC control 165. The counter 153 output (V_{CNTR}) provides a digital representation of the integrated charge at C_{J}. This digital representation is subsequently utilized to calculate the luminescence ratio, which, in turn, yields the measurement of the carbon dioxide level.

FIG. 5 shows a graphical representation of the integral values varied by the gaseous presence of carbon dioxide during operation of the sensing circuit 150 of FIG. 4. Referring to FIGS. 4 and 5, the operation of the CDC 168 can be analyzed in two phases, 501, 503. The first phase 501 happens when the sensing film 130 is being excited by a pulse of blue light from the light source 118. The modulation operation starts after the luminescence of the sensing films settles (after time t_{w1}), until then the integration node voltage V_{PD} is kept at V_{REF} via a switch (Φ_{RES1}). After the luminescence settles, the area under the I_{PD} curve, A₁, which is equivalent to the charge integrated at C_{J} during the integration, Q₁, can be found as:

$${A}_{1} = {Q}_{1} = ( {I}_{P D ( S E N )} + {I}_{P D ( R E F )} ) {T}_{INT} \qquad ( 1 )$$

where I_{PD(SEN)} is the carbon dioxide-sensitive component of the photocurrent, I_{PD}, and I_{PD(REF)} is the photocurrent contribution of the reference luminophores. TINT represents the duration of integration starting from the moment when luminescence stabilizes and continuing until the end of the excitation.

The second phase 503 starts upon the end of excitation. In this phase, the luminophores decay and the total luminescence is dominated by the reference component. The charge integrated during this phase, Q₂, is equivalent to the area under the I_{PD} curve during the decay, A₂:

$${A}_{2} = {Q}_{2} = {\int }_{{t}_{W 2}}^{{t}_{F 2}} {I}_{PD ( REF )} {e}^{\frac{- t}{τ}} dt \qquad ( 2 )$$

Where τ depicts the lifetime of the reference luminophores t_{W2} is configured to be roughly equivalent to the decay time of carbon dioxide-sensitive luminophores is the lifetime of the reference luminophores, ensuring that this component's influence is entirely disregarded during the integration process. t_{F2} is the duration of the decay of the reference luminophores which is about 5 τ. Since the lifetime of the carbon dioxide-sensitive luminophores is much shorter than the lifetime of the reference luminophores, Eq. 2 can be simplified as

$${A}_{2} = {Q}_{2} = \text{?} {I}_{P D ( R E F )} \qquad ( 3 )$$

$$\text{?} \text{indicates text missing or illegible when filed}$$

If we take the ratios of the charges Q₁ and Q₂:

$$\frac{{Q}_{1}}{{Q}_{2}} = \frac{( \text{?} + \text{?} ) {T}_{INT}}{{I}_{PD ( REF )}} \qquad ( 4 )$$

$$\text{?} \text{indicates text missing or illegible when filed}$$

Rearranging Eq. 4:

$$\frac{{I}_{PD ( SEN )}}{{I}_{PD ( REF )}} = \frac{{Q}_{1}}{{Q}_{2}} \frac{\text{?}}{{T}_{INT}} - 1 \qquad ( 5 )$$

$$\text{?} \text{indicates text missing or illegible when filed}$$

The CDC 168 converts charge, Q, to a digital number, N (at the output of the counter, V_{CNTR}). This conversion can be mathematically expressed as:

$${N}_{1 , 2} = \frac{{Q}_{1 , 2}}{{I}_{DAC , LSB} {T}_{CLK}}$$

where I_{DAC,LSB} is the least significant bit of the DAC and T_{CLK} is the clock period of the modulator. Replacing Q_{1,2} with N_{1,2}:

$$\frac{{I}_{PD ( SEN )}}{{I}_{PD ( REF )}} = \frac{{N}_{1}}{{N}_{2}} \frac{\text{?}}{{T}_{INT}} - 1$$

$$\text{?} \text{indicates text missing or illegible when filed}$$

As a result, it is possible to determine the luminescence ratio, represented as IPD_{(SEN)}/IPD_{(REF)}, by analyzing the CDC 168 outputs in two distinct phases, during 501 and after 503 the excitation. This ratio provides a quantitative measure of the carbon dioxide level to which the sensing film has been exposed. The computations may be performed in the sensing circuit 150 on the device, on the server 120, or any combination thereof. In other words, the sensing circuit 150 may transmit values to the server 120 where the analysis logic 126 computes the measurement of the sensed gas 132. The transmitter 114 allows any suitable Internet or other network 116 connection to communication with the server 120 for transmitting the output data 115 and allow computation of the carbon dioxide level or other sensed gas 132.

The operational logic of the sensing circuit 150, therefore, is for aggregating an intensity of each of the respective digital signals over an excitation phase and a decay phase, and computing a difference in the aggregated intensity for the respective digital signals for determining the concentration of the sensed gas. This includes aggregating the intensity corresponding to a sensory luminophore of the luminophore type, and aggregating the intensity corresponding to a reference luminophore of the luminophore type. Computing the difference further includes computing the luminescence ratio based on the intensity of both the sensory luminophores and the reference luminophores during the excitation phase, and on the intensity of the reference luminophores during the decay phase following excitation by the light source.

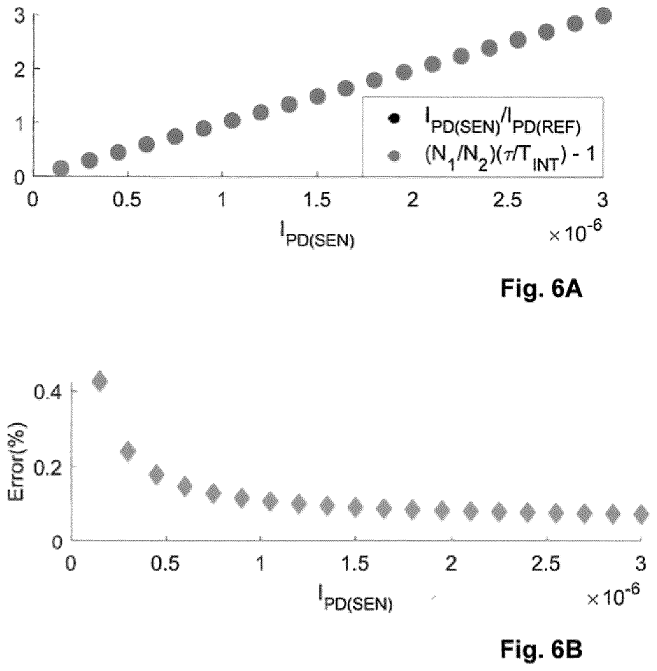

FIGS. 6A and 6B depict operational trials and verification of the disclosed approach. FIG. 6A presents the outcomes of behavioral simulations, during which I_{PD(SEN)} was adjusted within the range of 0.1 μA to 3 μA while maintaining I_{PD(REF)} at 1 μA, and the modulator's clock rate was set at 500 MHz. FIG. 6B shows the conversion error between the initial luminescence ratio and the values obtained from the CDC 168 output is less than 0.5%. It is worth emphasizing that the error is contingent on the operating frequency. As the frequency rises, the counter accumulates more counts, which in turn diminishes the conversional error.

Conventional approaches demonstrate alternate approaches, generally requiring heating elements and consequentially, associated circuit and power demands, or incorporate a tethered attachment to stationary processing units.

U.S. Pat. No. 4,930,506 shows an integrated sensor, equipped with a temperature-controlled heating unit, and employs a carbon dioxide measurement component based on the pH measurement in an electrolyte. The disclosed approach differs from an optical approach to detect carbon dioxide, whereas this device relies on the electrochemical principle of pH measurement in an electrolyte. Additionally, the '506 device necessitates the use of a heating element.

US 2010/0130842 shows an approach that depends on a local tissue blood flow that is factored in to the gaseous measurements. The sensor utilized in this setup comprises an electrochemical measurement apparatus for determining PtcCO₂. This electrochemical measurement device includes a micro-pH electrode and an Ag/AgCl reference electrode. Changes in the carbon dioxide levels at the skin surface induce pH variations in an electrolyte solution where both the micro-pH and Ag/AgCl reference electrodes are located. Unlike the disclosed approach, pH measurement is required for this wired sensor interface for a bulky bedside monitor.

U.S. Pat. No. 8,771,184 shows skin mounted electrodes on the patient for a variety of monitoring functions, and employs the conventional electrochemical method for measuring PtcCO₂, which requires a heating element.

U.S. Pat. No. 10,307,090 discloses an infrared-based sensor for detection of gas, including carbon dioxide. The sensor has a contact face which can be directed towards a measuring site. The sensor includes at least one radiation source, a measurement volume for receiving the gas to be measured, and detector for radiation transmitted. The disclosed approach incorporates no infrared sensing mediums.

U.S. Pat. No. 6,602,716 relies on a fluorescent response to fluorometrically assess a biological, chemical, or physical characteristic of a sample of two distinct luminescent materials.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A gaseous measurement device, comprising:
a sensing film having an emissive response to a sensed gas;
a light source, the sensing film having an emissive response to the light source based on a gaseous presence of the sensed gas;
a photodetector for receiving the emissive response from the sensing film; and
a sensing circuit responsive to the photodetector for computing:
i: a first digital signal indicative of the sensed gas; and
ii: a second digital signal indicative of a reference intensity, such that a concentration of the sensed gas is based on a ratio of the first digital signal and the second digital signal.

2. The device of claim 1 wherein:
the first digital signal is indicative of:
the emissive response from the sensing film including an emissive component based on the sensed gas and an emissive component based on a reference emission; and
the second digital signal is based on an emissive component based on the reference emission.

3. The device of claim 1 wherein the sensing film includes:
sensory luminophores responsive to the light source for emitting a response intensity based on a presence of the sensed gas; and
reference luminophores responsive to the light source for emitting a response intensity agnostic to the presence of the sensed gas.

4. The device of claim 1 wherein the sensing circuit includes a charge to digital converter, further comprising:
a comparator, the comparator connected to the photodetector for receiving a voltage signal indicative of the sensed gas;
a digital to analog converter (DAC) connected to an output of the comparator and configured for generating a feedback current responsive to an output from the comparator; and
a capacitor connected in parallel with the photodetector, the capacitor receiving the feedback current for integrating an aggregate signal received from the photodetector based on the emissive response.

5. The device of claim 4 further comprising:
a counter, the counter connected to the output of the comparator, the counter generating a count proportional to the emissive response from the sensing film based on both the sensed gas and the reference intensity.

6. The device of claim 1 wherein the sensing film further comprises:
sensory luminophores, the emissive response by the sensory luminophores having with an intensity varied by a presence of the sensed gas; and
reference luminophores,
wherein the first digital signal is indicative of a photodetector current responsive to a pulsed illumination based on an emissive intensity from both the sensory luminophores and the reference luminophores, and
the second digital signal is indicative of a photodetector current responsive to the pulsed illumination based on an emissive intensity from the reference luminophores.

7. The device of claim 6 wherein the sensory luminophores are responsive to a light at a predetermined wavelength for emitting the emissive response at a sensory wavelength with an intensity indicative of the gaseous presence of the sensed gas; and
the reference luminophores are responsive to the light for emitting the emissive response at a reference wavelength different than the sensory wavelength.

8. The device of claim 7 wherein the photodetector is responsive to light at both the sensory wavelength and the reference wavelength.

9. The device of claim 1 wherein the sensing film has a dual response based on a presence of the sensed gas, the dual response covering an emission spectra between 500-510 nm and between 595-605 nm.

10. The device of claim 1 wherein the sensing film is a carbon dioxide-sensitive film.

11. The device of claim 1 wherein the light source is configured to emit a blue light.

12. The device of claim 1 wherein the light source is configured to emit a light having a wavelength of 465 nm.

13. A method of detecting carbon dioxide, comprising:
disposing a sensing film in communication with a gaseous source, the sensing film having an emissive response to a sensed gas;
directing a light source at the sensing film, the sensing film responsive to the light source at a wavelength for generating the emissive response;
receiving the emissive response, the emissive response received at a photodetector from a plurality of luminophore types in the sensing film;
measuring a respective digital signal from each of the plurality of luminophore types based on the emissive response;
computing, based on a difference in the plurality of digital signals, a concentration of the sensed gas.

14. The method of claim 13 further comprising:
aggregating an intensity of each of the respective digital signals over an excitation phase and a decay phase; and
computing a difference in the aggregated intensity for the respective digital signals for determining the concentration of the sensed gas.

15. The method of claim 14 further comprising:
aggregating the intensity corresponding to a sensory luminophore of the luminophore type;
aggregating the intensity corresponding to a reference luminophore of the luminophore type,
wherein computing the difference further comprises computing a luminescence ratio based on the intensity of both the sensory luminophores and the reference luminophores during the excitation phase and the reference luminophores during the decay phase.

16. A gaseous measurement device, comprising:
a sensing film having an emissive response based on transcutaneous carbon dioxide (PtcCO₂);
a light source disposed for directing pulsed light at the sensing planar material;
a sensor for receiving re-emitted light from the sensing planar material; and
a monitoring circuit for identifying a carbon dioxide-sensitive component of a photocurrent emanating from the sensing film following settling of a luminescence from a pulse of the pulsed light.

17. The device of claim 16 wherein the light source irradiates a blue light.

18. The device of claim 16 wherein the monitoring circuit includes a charge-to-digital converter (CDC) used to derive carbon dioxide levels from luminescence data using a time-domain dual lifetime referencing approach.
